(19) United States
(12) Patent Application Publication
(10) Pub. No.: US 2002/0082684 A1
(43) Pub. Date: Jun. 27, 2002

(54) Intravascular prosthetic and method

(76) Inventor: David Mishaly, Tel Hashomer (IL)

Correspondence Address: Mark S. Leonardo, Esq., Brown Rudnick Freed & Gesmer, Box IP, 18th Floor, One Financial Center, Boston, MA, 02111, US

(21) Appl. No.: 09/951,010

(22) Filed: Sep. 12, 2001

Related U.S. Application Data
(60) Provisional application No. 60/235,034, filed on Sep. 25, 2000.

(51) Int. Cl. A61F 2/06

(52) U.S. Cl. 623/1.36

(57) ABSTRACT

An intravascular prosthetic graft is provided which includes a flexible tubular body having a first end defining at least one opening and a second end defining at least one opening. The flexible tubular body may be elongated and define a longitudinal axis. A first ring member is connected to the first end adjacent to the at least one opening of the first end. A second ring member is connected to the second end adjacent to the at least one opening of the second end. The flexible tubular body can be fabricated from a flexible material, such as, for example, DACRON®, TEFLON®, or other suitable material. The first and second ring members may be fabricated from a rigid material, such as metals or polymerics. A method of repairing at least a portion of an arterial system, such as, for example, treatment of an aortic aneurysm is disclosed. The method includes the steps of providing an intravascular prosthetic graft; determining a placement position for the intravascular prosthetic graft at an aneurysm site of the arterial system; introducing the intravascular prosthetic graft to the arterial system; delivering the intravascular prosthetic graft to the aneurysm site; positioning the intravascular prosthetic graft for repairing a diseased portion of an aorta at the placement position; expanding the intravascular prosthetic graft to substantially conform to an inner surface of the aorta; and fixing the intravascular prosthetic graft at the placement position. The step of fixing may include tying a suture about the exterior of the aorta. The method may further include the step of suturing the aorta to the intravascular prosthetic graft from exterior to the aorta.

### CROSS-REFERENCE TO RELATED APPLICATIONS

This Patent Application claims the benefit of U.S. Provisional Application No. 60/235,034 filed Sep. 25, 2000, the contents of which are incorporated herein by reference in its entirety.

### BACKGROUND

1. Technical Field

The present disclosure relates generally to vascular repair of body vessels, and more particularly, to an apparatus and method for treating aortic aneurysms, such as those which involve the ascending aorta, with an expandable prosthetic graft.

2. Description of Related Art

A leading cause of disability and death has been damage to a portion of the arterial system which carries blood from the heart to the various parts of the body. This has been a particular problem with regard to aneurysms in the aorta, which is the main trunk of the arterial system and which conveys blood from the left ventricle of the heart to all of the body except the lungs. Further, diseases of the aorta have become a major issue as a result of advancements in cardiac surgery and human life longevity. Severe arterial sclerosis, a severely calcified aorta or other indications have necessitated the replacement of portions of the aorta.

Aneurysms are typically characterized by diseased or damaged blood vessels which often cause weakening of the vessel wall, whereby a blood vessel and especially an artery, have a section of blood-filled dilation. Left untreated, the aneurysm will eventually rupture with ensuing fatal hemorrhaging in a very short time period. High mortality associated with rupturing led the state of the art to uncover procedures for treating the aneurysm.

In recent years, a great advance has been made in the medical, and particularly, in the surgical art, whereby the portion of the arterial system in which the aneurysm, calcification, etc., occurs is cut away and resected and a tubular prosthetic is substituted for the resected segment. This prosthetic, typically, is a synthetic or graft, corresponding in diameter to the resected arterial segment. The tubular prosthetic graft, after it is set in place, is then sutured at its opposite ends to the adjacent edges of an artery defining the space resulting from the resected segment. The prosthetic typically is a synthetic graft usually fabricated from, for example, DACRON®, TEFLON®, or other suitable material.

Aneurysms, calcification, etc., are treated, for example, by open heart surgery and less invasive techniques. Open heart surgery can be performed to replace portions of the arterial system, such as, for example, the ascending aorta, aortic arch, etc. Open heart surgery, however, is a major undertaking with associated high risks. This procedure includes deep cooling of the body, deep hypothermic circulatory arrest and cardiac ischemia. These factors have resulted in high mortality and morbidity rates, as well as high associated costs of the procedures.

Currently, some arterial aneurysms are repaired by less invasive techniques. Although these less invasive procedures have constituted a major development in the field of medicine, various drawbacks have arisen. Based on these drawbacks, less invasive techniques have been limited to repair of the distal part of the aorta.

Typically, a minimally invasive technique for treating an aneurysm introduces a prosthetic graft which is deployed into an arterial system remote from an aneurysm site via a catheter traveling through the arterial system. The prosthetic graft must be manipulated through the arterial system and fixed to the vessel at the aneurysm site remotely. Remote manipulation of the prosthetic graft can be very difficult, causing kinking or twisting of the prosthetic graft.

In particular, in treating an ascending aortic aneurysm, the minimally invasive techniques suffer from many disadvantages. For example, when using minimally invasive techniques such as percutaneous repair, the aortic neck, proximal to the aneurysm and typically not effected by the aneurysm, is not long enough to support an endovascular device. Further, while treating an ascending aortic aneurysm involving the aortic arch, the curvature of the aorta prevents a stented graft from proper placement therein due to the stented graft being capable of positioning with a vessel portion having only a minor curvature. Also, the side branches or neck vessels, adjacent to the aorta, make positioning of the prosthetic graft a complex procedure.

Typically, during a minimally invasive procedure, the prosthetic graft is delivered intraluminally to the aneurysm site. The prosthetic graft is secured to the remaining vessel wall by an endovascular expander, such as a balloon, stent, etc., that expands the graft and vessel, causing the graft to contact and adhere to the remaining vessel wall.

Major drawbacks of the absence of a normal proximal vessel neck portion for fixating and/or anchoring the prosthetic graft with, migrations and periprosthetic losses. Upon withdrawal of the endovascular expander, the prosthesis graft disengages from the remaining vessel wall and tends to migrate to a location away from the aneurysm site. The migration and movement of the disengaged aortic graft can obstruct the effected vessel. The migration and movement of the prosthetic graft requires further treatment of the patient to remove the failed attempt to attach the prosthetic graft to the remaining vessel wall. Further treatment may include major surgery that is hazardous and traumatic to a patient.

Accordingly, a need exists for an expandable intravascular prosthetic graft and method that repairs at least a portion of an arterial system and in particular, a diseased aorta by employing a minimal risk technique that reduces the associated dangers to a patient. Preferably, the prosthetic graft and minimal risk technique facilitate easy maneuverability through an arterial system to reduce complexity of arterial repair. Desirably, the prosthetic graft replaces at least a portion of the ascending aorta, the arch of the aorta, the innominate artery and/or the descending aorta.

It is, therefore, an object of the present disclosure to overcome the disadvantages of the prior art by providing an expandable intravascular prosthetic graft and method that repairs at least a portion of an arterial system and, preferably, a diseased aorta, by employing a minimal risk technique that reduces the associated dangers to a patient.

It is a further object of the present disclosure to provide an expandable intravascular prosthetic graft and method that facilitate easy maneuverability through an arterial system to reduce complexity of arterial repair.

It is another object of the present disclosure to provide an expandable intravascular prosthetic graft and method that repairs at least a portion of the ascending aorta, the arch of the aorta, the innominate artery and/or the descending aorta.

It is yet another object of the present disclosure to provide an expandable intravascular prosthetic graft and method which reduce high mortality and high morbidity rates and facilitates human life longevity.

It is still yet another object of the present disclosure to provide an expandable intravascular prosthetic graft and method which are easily and efficiently fabricated to reduce associated costs of manufacture and surgical procedure.

Objects and advantages of the present disclosure, set forth in part herein and in part will be obvious therefrom, achieving the intended purposes, objects, and advantages through a new, useful and non-obvious configuration of component elements at reasonable costs to manufacture and by employing readily available materials. The various embodiments contemplated are gleaned from the present disclosure and realized and attained by means of the instrumentalities and combinations pointed out herein.

### SUMMARY

Accordingly, the present disclosure is directed to an expandable intravascular prosthetic graft and related methods designed to repair a diseased ascending aorta and/or aortic arch. It is envisioned that the expandable intravascular prosthetic graft is anastomosed to the native vessel by sutureless rings. The replacement procedure can be completed without requiring a heart lung machine or opening the thoracic aorta.

The present disclosure provides the expandable intravascular prosthetic graft and method that repairs at least a portion of an arterial system and, preferably, a diseased aorta that employs a minimal risk technique that reduces the associated dangers to a patient. Preferably, the prosthetic graft and minimal risk technique facilitate easy maneuverability with an arterial system to reduce complexity of arterial repair. Desirably, the prosthetic graft repairs at least a portion of the ascending aorta, the arch of the aorta, the innominate artery and/or the descending aorta.

In one particular embodiment, in accordance with the present disclosure, an intravascular prosthetic graft is provided which includes a flexible tubular body having a first end defining at least one opening and a second end defining at least one opening. The flexible tubular body may be elongated and define a longitudinal axis. A first ring member is connected to the first end adjacent to the at least one opening of the first end. A second ring member is connected to the second end adjacent to the at least one opening of the second end. The flexible tubular body can be fabricated from a flexible material, such as, for example, DACRON®, TEFLON®, or other suitable material. Alternatively, the flexible tubular body may be fabricated from a semi-rigid material. The first and second ring members may be fabricated from a rigid material, such as metals or polymerics. Alternatively, the first and second ring members can be constructed from a semi-rigid material.

The second ring member may be connected to the second end adjacent to the at least one opening of the second end and oriented at a transverse axis relative to the longitudinal axis of the flexible tubular body. The first and second ring members may define a center groove disposed about their respective circumferences.

In an alternate embodiment, the intravascular prosthetic includes a third ring member connected to the second end of the flexible tubular body adjacent to a third opening of the second end.

In another alternate embodiment, the intravascular prosthetic graft includes a flexible tubular body and a first end defining an opening and a second end defining an opening. The flexible tubular body includes at least one lateral opening. A first ring member is connected to the first end adjacent to the opening of the first end and a second ring member is connected to the second end adjacent to the opening of the second end. A third ring member is connected to a lateral opening of the flexible tubular body. The flexible tubular body may include three lateral openings whereby the third ring member, a fourth ring member, and a fifth ring member are connected to the lateral openings. A sixth ring member may be connected to the flexible tubular body between the first and second ends thereof.

A method of repairing at least a portion of an arterial system, such as, for example, treatment of an aortic aneurysm is disclosed. The method includes the steps of providing an intravascular prosthetic graft, similar to those described above; determining a placement position for the intravascular prosthetic graft at an aneurysm site of the arterial system; introducing the intravascular prosthetic graft to the arterial system; delivering the intravascular prosthetic graft to the aneurysm site; positioning the intravascular prosthetic graft for repairing a diseased portion of an aorta at the placement position; expanding the intravascular prosthetic graft to substantially conform to an inner surface of the aorta; and fixing the intravascular prosthetic graft at the placement position. The step of fixing may include tying a suture about the exterior of the aorta. The method may further include the step of suturing the aorta to the intravascular prosthetic graft from exterior to the aorta.

### BRIEF DESCRIPTION OF THE DRAWINGS

The objects and features of the present disclosure, which are believed to be novel, are set forth with particularity in the present disclosure. The present disclosure, both as to its organization and manner of operation, together with further objectives and advantages, may best be understood by reference to the following description taken in connection with the accompanying drawings, in which:

FIG. 1 is a side perspective view of an intravascular prosthetic graft, in accordance with one embodiment of the present disclosure;

FIG. 2 is a side perspective view of an alternate embodiment of the intravascular prosthetic graft shown in FIG. 1;

FIG. 3 is a side perspective view of a ring member of the intravascular prosthetic graft shown in FIG. 1;

FIG. 4 is a perspective view of an arterial system and the intravascular prosthetic grafts shown in FIGS. 1 and 2;

FIG. 5 is a perspective view of the arterial system shown in FIG. 4, and the intravascular prosthetic graft shown in FIG. 2, being delivered to an aneurysm site;

FIG. 6 is a perspective view of the arterial system shown in FIG. 4, and the intravascular prosthetic graft shown in FIG. 2, at the aneurysm site;

FIG. 7 is a perspective view of the arterial system shown in FIG. 4, and the intravascular prosthetic graft shown in FIG. 2, at the aneurysm site after expansion;

FIG. 8 is an enlarged detail perspective view of a portion of the arterial system shown in FIG. 4, and the intravascular prosthetic graft shown in FIG. 1 being fixed to the aorta;

FIG. 9 is an enlarged detail perspective view of a portion of the arterial system shown in FIG. 4, and the intravascular prosthetic graft shown in FIG. 2 being fixed to an aorta;

FIG. 10 is an enlarged detail perspective view of an alternate embodiment of the intravascular prosthetic graft shown in FIG. 1, being fixed to a portion of the arterial system shown in FIG. 4; and

FIG. 11 is an enlarged detail perspective view of another alternate embodiment of the intravascular prosthetic graft shown in FIG. 1, being fixed to a portion of the arterial system shown in FIG. 4.

### DETAILED DESCRIPTION OF THE EXEMPLARY EMBODIMENTS

The exemplary embodiments of an expandable intravascular prosthetic graft and methods of operation are described in terms of treating diseased vessels, such as, for example, repairing aortic aneurysms to remove diseased portions of the ascending aorta and aortic arch, including the proximal portion of one or more associated neck vessels, using a minimal risk technique. It is contemplated that the expandable intravascular prosthetic graft can be anastomosed to the arterial system by sutureless rings. The minimal risk technique facilitates easy maneuverability of the intravascular prosthetic graft with an arterial system to reduce complexity of arterial repair.

The disclosed intravascular prosthetic graft and method advantageously avoid the disadvantages of open heart surgery such as, for example, requiring a heart lung machine or opening the thoracic aorta. Drawbacks of minimally invasive techniques, such as, for example, migration, periprosthetic leakage and complex positioning procedures are also avoided. Further, the intravascular prosthetic graft and methods disclosed advantageously facilitate a fixed and dependable repair of a diseased vessel portion.

Treatment of an aortic aneurysm includes providing the intravascular prosthetic graft and delivering the intravascular prosthetic graft to the aneurysm site via introduction to the arterial system through the femoral artery adjacent the groin of a patient. The intravascular prosthetic graft is introduced through the arterial system to the aneurysm site for treatment of a diseased arterial portion. The intravascular prosthetic graft may be utilized in the thoracic aorta, and can be used to repair thoracic aneurysms or thoracic dissecting aneurysms. Further, the intravascular prosthetic graft may also treat vascular trauma, arteriosclerosis, calcification and other obstructive diseases with various prosthesis. Accordingly, the use of the term aortic aneurysm in the present disclosure is intended to relate to and mean both thoracic aneurysms, abdominal aortic aneurysms and related vessel diseases.

In the discussion which follows, the term “proximal” will refer to the portion of a structure which is closer to a heart, while the term “distal” will refer to the portion which is further from the heart . As used herein the term “patient” refers to a subject that is being treated for an aneurysm, vessel diseases, etc. According to the present disclosure, the term “surgeon” refers to an individual performing the procedure for treating an aneurysm, vessel disease, etc., and may include support personnel.

The following discussion includes a description of the intravascular prosthetic graft, followed by a description of the methods of employing the intravascular prosthetic graft during a surgical procedure, in accordance with the present disclosure. Reference will now be made in detail to the exemplary embodiments of the present disclosure, which are illustrated in the accompanying FIGURES.

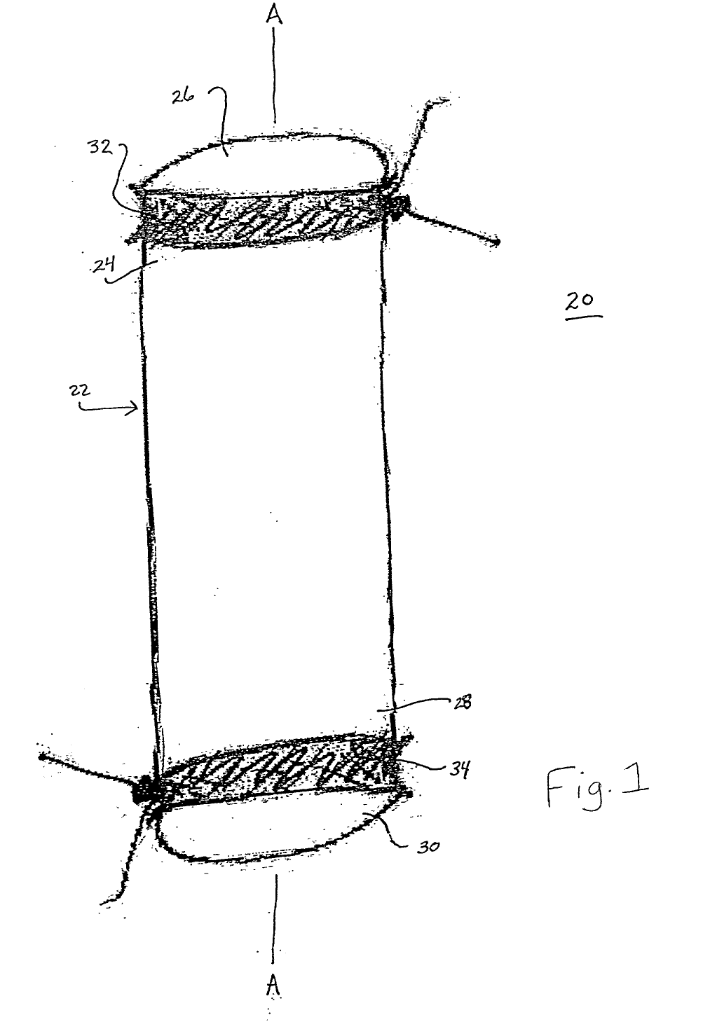

Turning now to the FIGURES wherein like components are designated by like reference numerals throughout the several views. Referring initially to FIG. 1, there is illustrated an intravascular prosthetic graft 20, constructed in accordance with the principals of the present disclosure, which includes a flexible tubular body 22 that is elongated and defines a longitudinal axis A. Flexible tubular body 22 is substantially cylindrical and manufactured from a material which allows flexible tubular body 22 to substantially conform to the interior walls of a blood vessel such as, for example, an artery (not shown). Flexible tubular body 22 is constructed from a flexible material, such as, for example, polytetrafluroethylene, such as TEFLON® Flexible tubular body 22 may also be woven from a synthetic polyester such as DACRON®. One skilled in the art, however, will realize that other materials and fabrication methods suitable for body vessel surgical applications, in accordance with the present disclosure also would be appropriate.

Flexible tubular body 22 is constructed such that intravascular prosthetic graft 20 is easily maneuverable within a vessel, including portions of the vessel which may have sharp curvatures, etc., without becoming kinked or twisted. Flexible tubular body 22 is also expandable by, such as, for example, a balloon, stent, etc., such that flexible tubular body 22 substantially conforms to the inner surface of a vessel, as will be discussed below.

Flexible tubular body 22 has a first end 24 defining an opening 26. Opening 26 is substantially cylindrical and flexible for substantially conforming to the inner surface of an artery. Opening 26 is configured for the flow of fluid, such as blood, therethrough. A second end 28 defines an opening 30 which similarly has a substantially cylindrical configuration, conformable to the inner surface of a vessel and configured for the flow of blood therethrough. It is contemplated that openings 26 and 30 are similarly configured for being fixed adjacent to a diseased portion of a vessel during a surgical procedure.

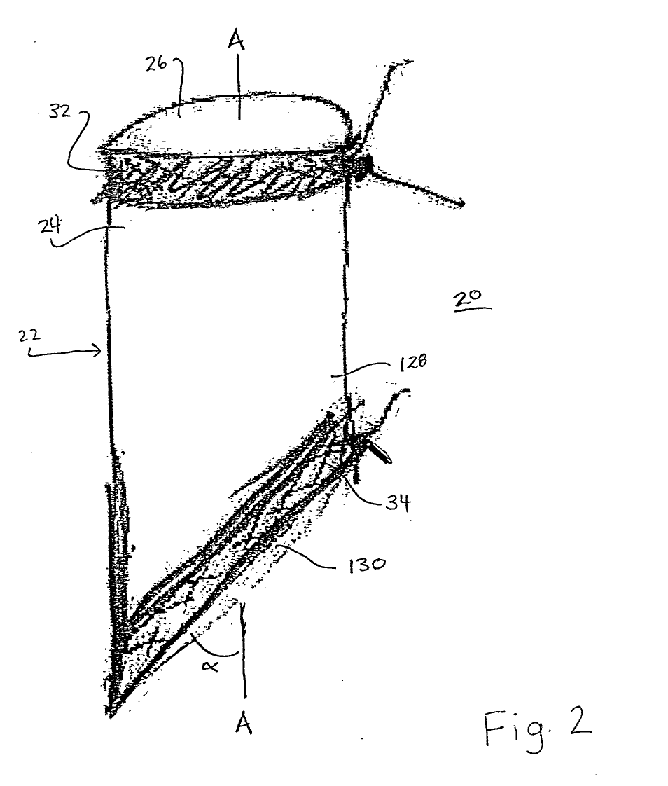

In an alternate embodiment, as shown in FIG. 2, intravascular prosthetic graft 20 includes a second end 128 defining an opening 130, similar to that discussed above. Opening 130 lies in a plane transverse to longitudinal axis A at an angular orientation α relative to longitudinal axis A. The orientation of opening 130 advantageously facilitates facile maneuverability of intravascular prosthetic graft 20 within a vessel. For example, during repair of an ascending aorta, the transverse orientation of opening 130 facilitates arterial repair of areas of the aortic arch involved in the pathology associated with the ascending aorta which may be referred to as the hemiarch technique. It is contemplated that the hemiarch technique comprises about 80% of an aortic surgical procedure. It is contemplated that opening 130 may be oriented at various angular orientations α relative to longitudinal axis A, according to the particular surgical application, pathologic condition and/or preference of the surgeon.

With continued reference to FIGS. 1 and 2, a first ring member 32 is connected to first end 24 adjacent to opening 26 of first end 24. A second ring member 34 is connected to second end 28, 128 adjacent to opening 30, 130 of second end 28, 128. As shown in FIG. 2, second ring member 34 is oriented corresponding to the orientation of opening 130, at angular orientation α. It is contemplated that ring members 32 and 34 may be variously oriented with openings 26, 30, 130. First ring member 32 and second ring member 34 are integrally assembled with flexible tubular body 22 and have a substantially cylindrical configuration for conforming to the inner surface of a vessel. First ring member 32 and second ring member 34 are designed to have a first configuration, such as, for example, a non-expanded state for introduction into an arterial system and delivery to an aneurysm site, as will be discussed below. First ring member 32 and second ring member 34 are expandable to a second configuration, such as, for example, an expanded state, whereby first ring member 32 and second ring member 34 substantially conform to the inner surfaces of a vessel.

First ring member 32 and second ring member 34 are constructed from a rigid or semi-rigid material, such as, for example, stainless steel, in a particular configuration which facilitates expansion of ring members 32 and 34 from the non-expanded state to the expanded state. It is contemplated that ring members 32 and 34 may be fabricated from a material suitable for surgical applications, in accordance with the present disclosure, such as polymerics or other metals, having the inherent characteristics of strength, non-corrosiveness, etc. For example, aluminum may be used and anodized for non-corrosive characteristics. Alternatively, ring members 32 and 34 may be self-expanding by using a shape memory material, such as, for example, Nitinol, etc. One skilled in the art, however, will realize that other materials and fabrication methods suitable for surgical applications, in accordance with the present disclosure, also would be appropriate.

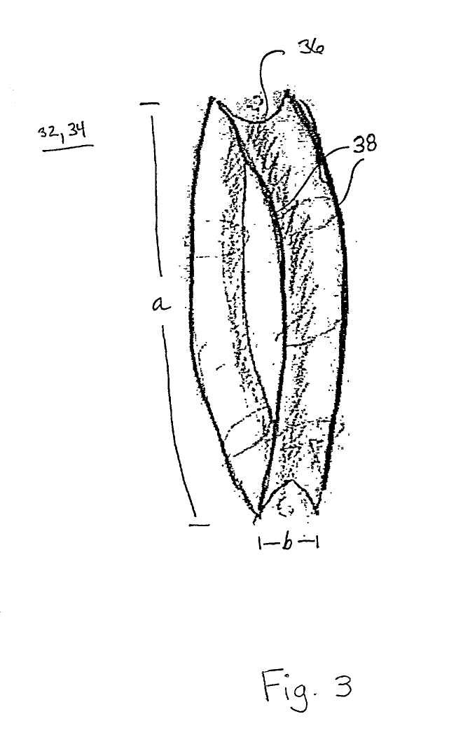

Referring to FIG. 3, ring members 32 and 34 define a substantially convex groove 36 in an outer surface thereof about their respective circumferences between ends 38. Groove 36 is formed about the respective circumferences of ring members 32 and 34 to advantageously facilitate receipt of a suture tie (not shown) therein. Receipt of the suture tie within groove 36 facilitates fixing the inner surface of a vessel to intravascular prosthetic graft 20, discussed below. It is contemplated that groove 36 may be formed about a portion of ring members 32 and 34 sufficient to maintain the suture tie therein.

Ring members 32 and 34 have a diameter a and groove 36 has a thickness b. It is contemplated that diameter a and thickness b may be variously dimensioned according to the requirements of a particular surgical application and/or preference of the surgeon. In the expanded state, ring members 32 and 34 of intravascular prosthetic graft 20 engage the inner surface of a vessel, such that an outer surface of intravascular prosthetic graft 20 sealingly engages the inner surface of the vessel. This facilitates blood flow through intravascular prosthetic graft 20 such that blood flow will not circumvent intravascular prosthetic graft 20 thereby preventing fluid leakage.

It is contemplated that groove 36 may have a substantially acute or a substantially obtuse convex configuration according to the particular surgical application. It is further contemplated that when intravascular prosthetic graft 20 is in the expanded state, diameter a has a dimension of 30 millimeters and thickness b has a dimension of 6 millimeters. It is envisioned that groove 36 may be formed equidistantly from ends 38 of ring members 32 and 34. Groove 36 may also be variously spaced about the outer surface of ring members 32 and 34 and formed adjacent to one of ends 38 according to the particular surgical application.

The operation of intravascular prosthetic graft 20 during an aneurysmal treatment procedure will now be described. The surgical procedure described contemplates repairing aortic aneurysms by a minimal risk technique and replacing at least a portion of an aorta. It is envisioned that other portions of an arterial system may also be repaired. It is further envisioned that intravascular prosthetic graft 20 may be utilized for repair of the ascending aorta and related portions, treating severe arteriosclerosis and/or a severely calcified aorta and other indications to avoid the associated dangers to a patient.

The method and minimal risk technique described using intravascular prosthetic graft 20 advantageously repairs a diseased vessel without requiring the use of a heart/lung machine. The method provides facile maneuverability of intravascular prosthetic graft 20 within an artery and fixes intravascular prosthetic graft 20 to an inner surface of the artery. Modified methods for replacing various portions of a vessel are also contemplated and can be gleaned from the discussion below.

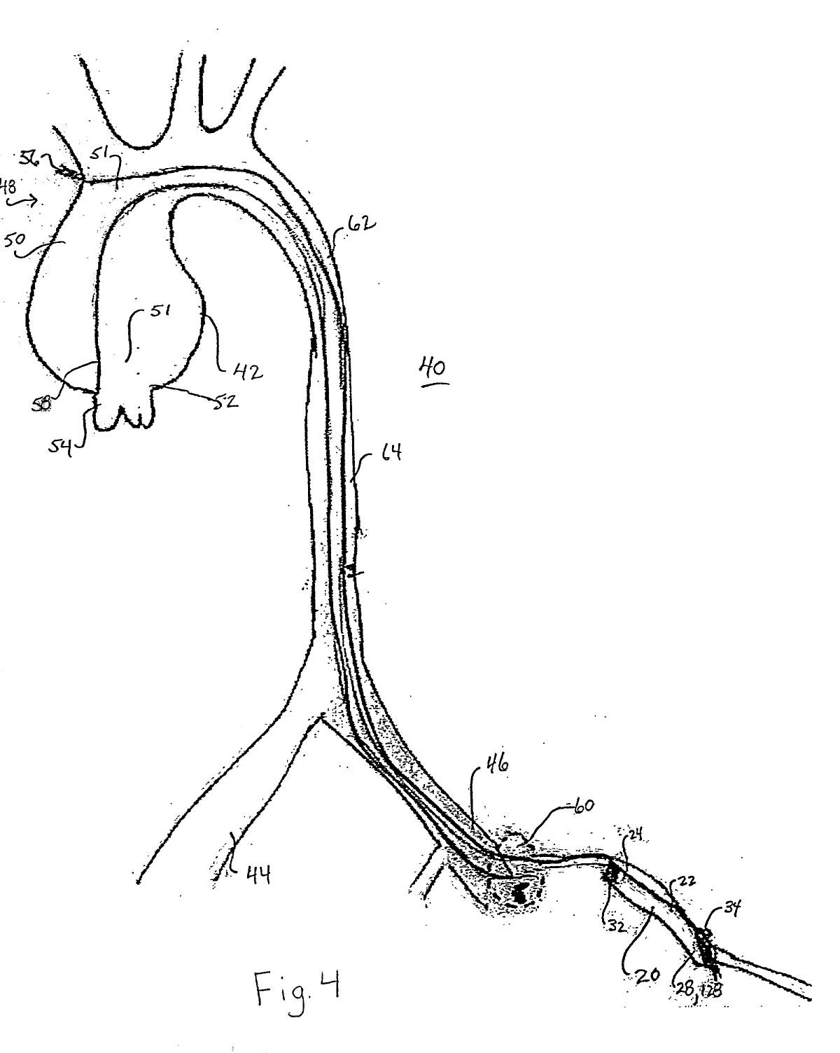

Referring to FIGS. 4-9, an arterial system 40 having a diseased vessel portion, such as, for example, an aneurysm 42 is shown. The method of repairing at least a portion of arterial system 40, such as, for example, treatment of aortic aneurysm 42 includes the steps of providing intravascular prosthetic graft 20, similar to that described above; determining a placement position, such as, for example, polarity sites 51, for intravascular prosthetic graft 20 at the site of aneurysm 42; introducing intravascular prosthetic graft 20 to arterial system 40; delivering intravascular prosthetic graft 20 to the site of aneurysm 42; positioning intravascular prosthetic graft 20 for repairing a diseased portion of an aorta 48 at polarity sites 51; expanding intravascular prosthetic graft 20 to substantially conform to an inner surface of aorta 48; and fixing intravascular prosthetic graft 20 to the inner surface of aorta 48 at polarity sites 51.

Referring to FIG. 4, arterial system 40 includes a right iliac artery 44 and a left iliac artery 46. To determine polarity sites 51 for intravascular prosthetic graft 20, the chest of the patient is open by midsternotomy. Aorta 48 is dissected and an ascending aorta 50 separated from a pulmonary artery (not shown). A sinotubular junction 52 is located above aortic sinuses 54. The above steps are performed to facilitate determination of polarity sites 51 whereby first ring member 32 and second ring member 34 of intravascular prosthetic graft 20 will be fixed to portions of aorta 48.

Intravascular prosthetic graft 20 is introduced to arterial system 40. A guide wire 56 is inserted via the chest opening adjacent polarity sites 51, i.e., the portion of aorta 48 to be replaced. A second guide wire 58 is similarly inserted within aorta 48.

In an alternate embodiment, polarity sites 51 may be determined and guide wires 56, 58 may be introduced adjacent polarity sites 51 by employing minimally invasive and laparascopic apparatus and techniques. For example, minimally invasive techniques that use instrumentation (not shown) such as, a cannula, trocar, etc., that is inserted into the body and/or a body cavity through a small endoscopic incision (not shown) for performing a surgical procedure are contemplated. Further instrumentation, such as, those for making incisions, manipulating organs, body vessels, etc., may be introduced adjacent to the site of aneurysm 42. Such minimally invasive instrumentation would facilitate, for example, determination of polarity sites 51, introduction of guide wires 56 and 58 and fixing of the inner surface of aorta 48 to an outer surface of intravascular prosthetic graft 20, as will be discussed below.

Referring back now to FIG. 4, the surgeon (not shown) makes an incision within right iliac artery 46 at an opening site 60. Guide wires 56 and 58 are advanced from polarity sites 51, through a descending aorta 62, a thoracic artery 64, right iliac artery 46 to opening site 60. Intravascular prosthetic graft 20 is in the non-expanded state, described above.

Guide wires 56 and 58 are fastened to intravascular prosthetic graft 20. Guide wire 58 is attached to first end 24 of flexible tubular body 22 and guide wire 56 is attached to second end 28, 128, respectively. Guide wires 56 and 58 may be attached to intravascular prosthetic graft 20 by hooks (not shown) which may be attachable and removable, before and after the surgical procedure. A hook attachment may also be removable in a breakaway arrangement inside and/or exterior to intravascular prosthetic graft 20. Guide wires 56 and 58 may also be adhesively attached to intravascular prosthetic graft 20 or, alternatively, may be connected by a suture stitch which may be broken away from intravascular prosthetic graft 20 upon completion of the procedure. One skilled in the art, however, will realize that other attachment methods may be suitable in accordance with the present disclosure.

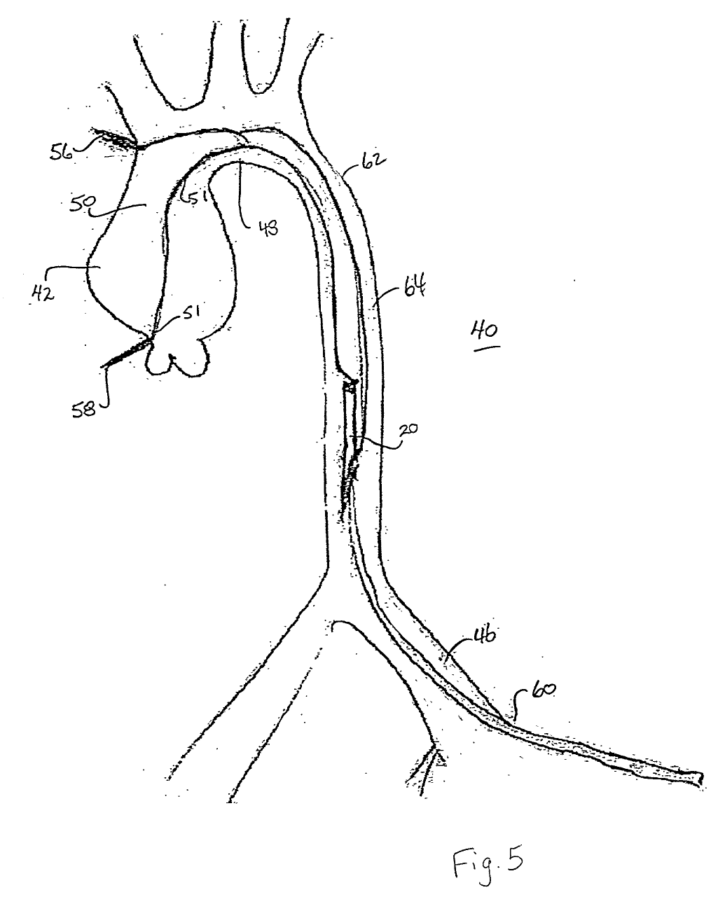

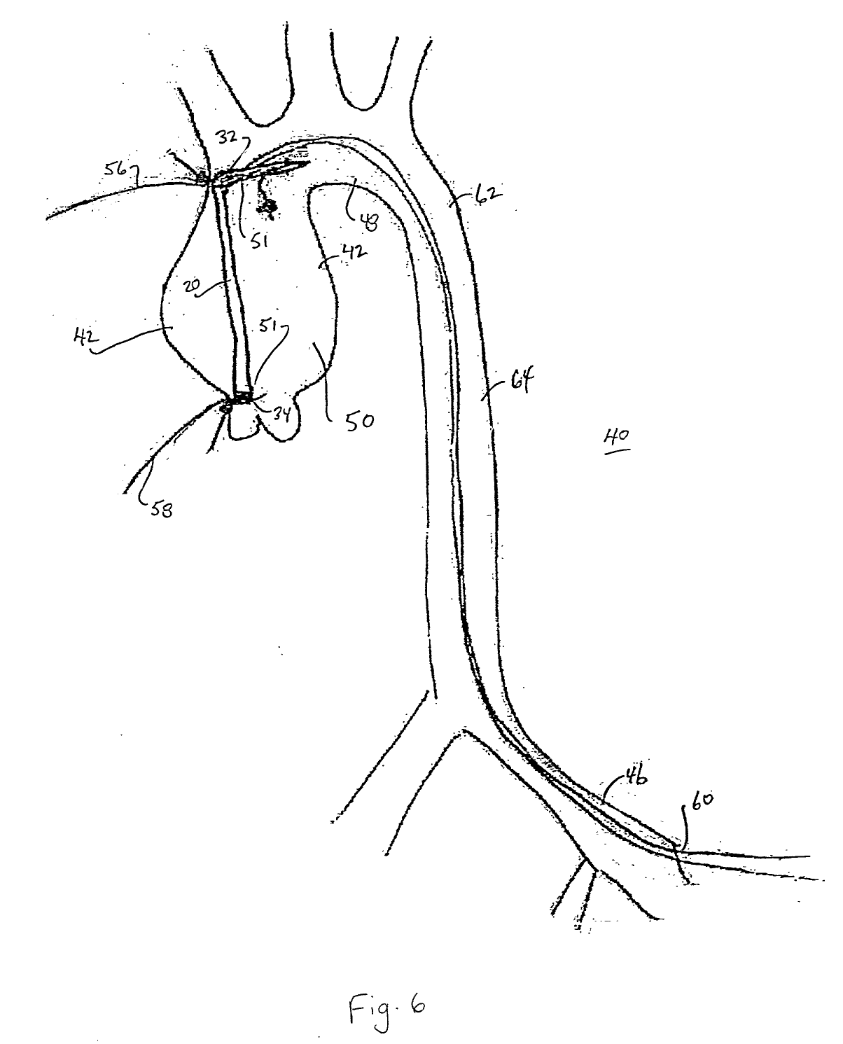

Referring to FIGS. 5 and 6, guide wires 56 and 58, attached to intravascular prosthetic graft 20 are then pulled back through arterial system 40 such that intravascular prosthetic graft 20 is introduced into opening site 60 through iliac artery 46, thoracic artery 64, descending aorta 62 into and adjacent the area where ascending aorta 50 will be replaced such that aneurysm 42 will be treated.

The connection of guide wires 56 and 58 to intravascular prosthetic graft 20 and corresponding travel of intravascular prosthetic graft 20 to the site of aneurysm 42 facilitates positioning of intravascular prosthetic graft 20 adjacent polarity sites 51. First ring member 32 and second ring member 34 are properly positioned with polarity sites 51 to sit superior and not inferior within ascending aorta 50. The cooperation of ring members 32 and 34 and polarity sites 51 advantageously avoids kinking or twisting of intravascular prosthetic graft 20 adjacent aneurysm 42.

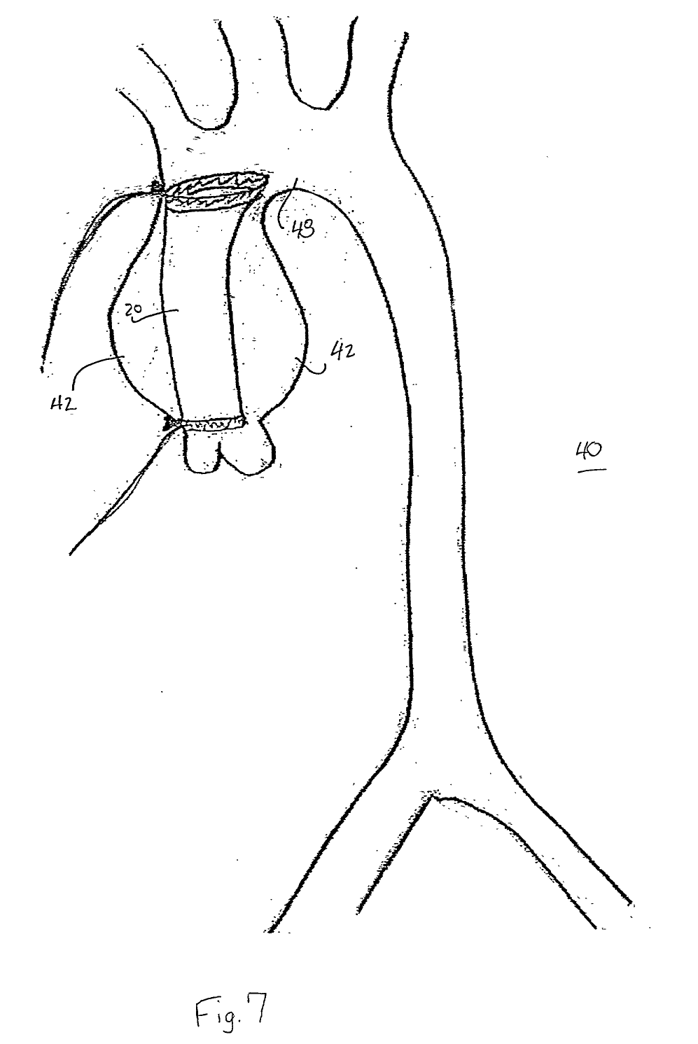

Referring to FIGS. 6 and 7, intravascular prosthetic graft 20 is expanded to the expanded state, as discussed above. Intravascular prosthetic graft 20 is expanded by, for example, a balloon, stent, etc., (not shown) or alternatively may be self-expanding by, a shape memory material, such as, for example, Nitinol, etc. Intravascular prosthetic graft 20 is in the expanded state to substantially conform to the inner surfaces of aorta 48. Intravascular prosthetic 20 has a smaller diameter than aneurysm 42, such that intravascular prosthetic graft 20 does not substantially dilate aorta 48 above its natural diameter.

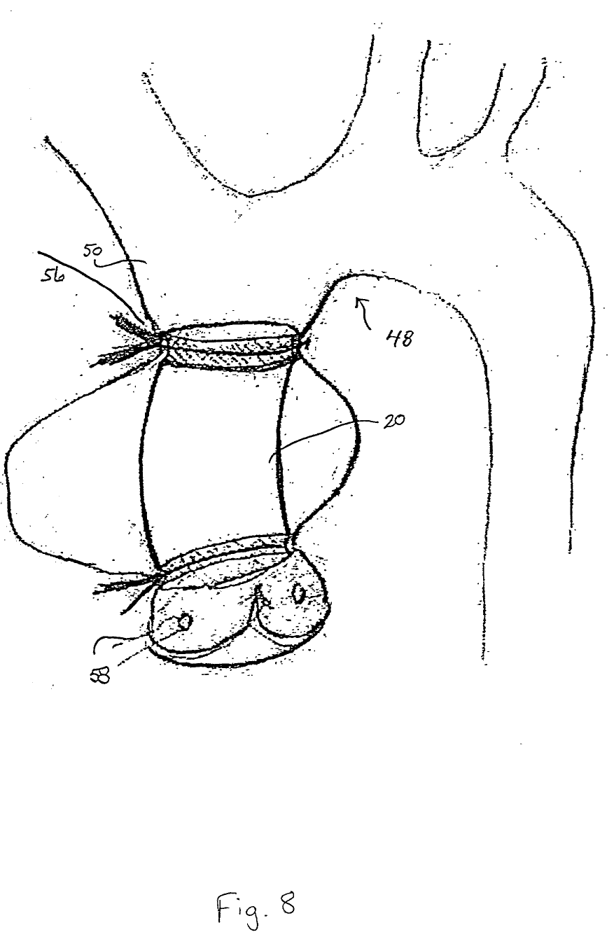

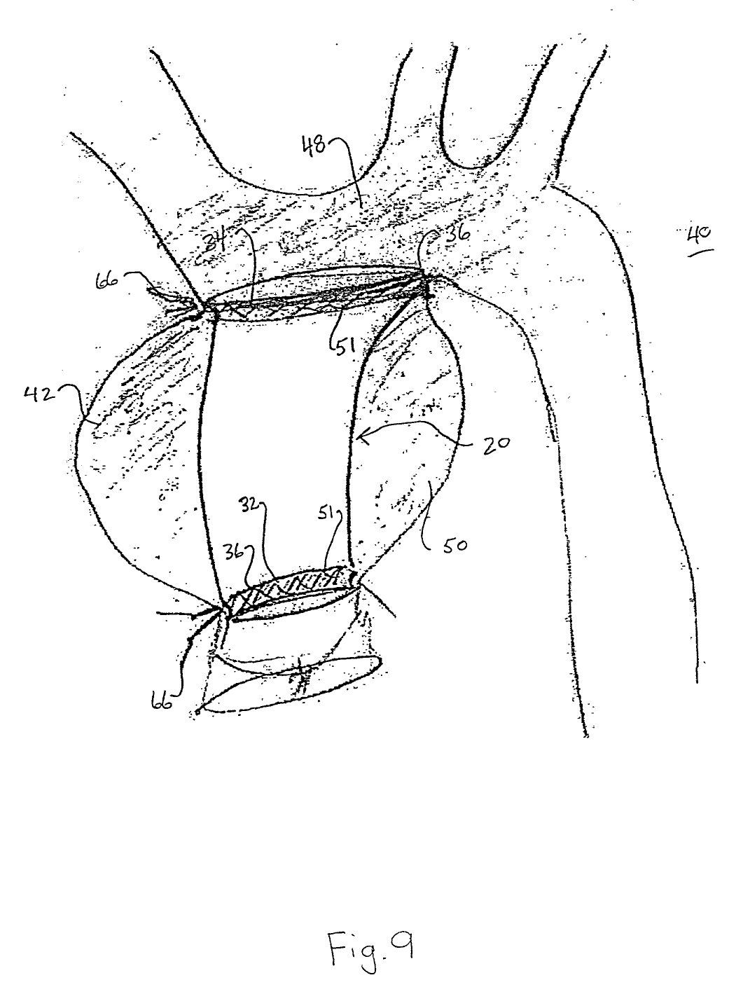

Referring to FIGS. 8 and 9, with prosthetic graft 20 in position, a ligature/tie 66 is disposed about and engages the outside of aorta 48 within the area defined by the corresponding grooves 36 (FIG. 3). Ligature/tie 66 is tied in place to fix prosthetic graft 20 with aorta 48, as indicated in FIGS. 8-11. Grooves 36 cause ligature 66, when tightly engaged, to be positioned within the area defined between ends 38 of grooves 36 in aorta. Ends 38 (FIG. 3) form ridges in aorta 48 which prevent lateral slippage of ligature 66.

Referring to FIG. 8, guide wires 56 and 58 are removed, via the chest opening, such that intravascular prosthetic graft 20 may be fixed to the inner surface of portions of aorta 48 adjacent to the diseased portions of ascending aorta 50. It is contemplated that guide wires 56 and 58 may remain until the procedure is complete, or may comprise disintegrating suture which can remain in the patient.

Referring to FIG. 9, ligature 66 are manipulated, via the chest opening, exterior to aorta 48 adjacent polarity sites 51 and the portion of ascending aorta 50 to be repaired. Ligature 66 are tied at polarity sites 51 such that ligature 66 are tied exterior to the portion of ascending aorta 50 adjacent polarity sites 51. As ligature 66 are received by grooves 36 of first ring member 32 and second ring member 34, ligature 66 engage an exterior surface of aorta 48 forcing aorta 48 to similarly engage grooves 36. The inner surface of aorta 48 engages and become fixed with ring members 32 and 34. This reduces the vessel size of aorta 48 to the size of ring members 32 and 34. Blood is thereby forced into intravascular prosthetic graft 20 and leakage is prevented.

Alternatively, after fixation of aorta 50 with intravascular prosthetic graft 20, sutures may be applied adjacent to first ring member 32 and second ring member 34 to reinforce the attachment of aorta 48 and intravascular prosthetic graft 20. Sutures may also be applied to holes (not shown) alternatively formed within ring members 32, 34.

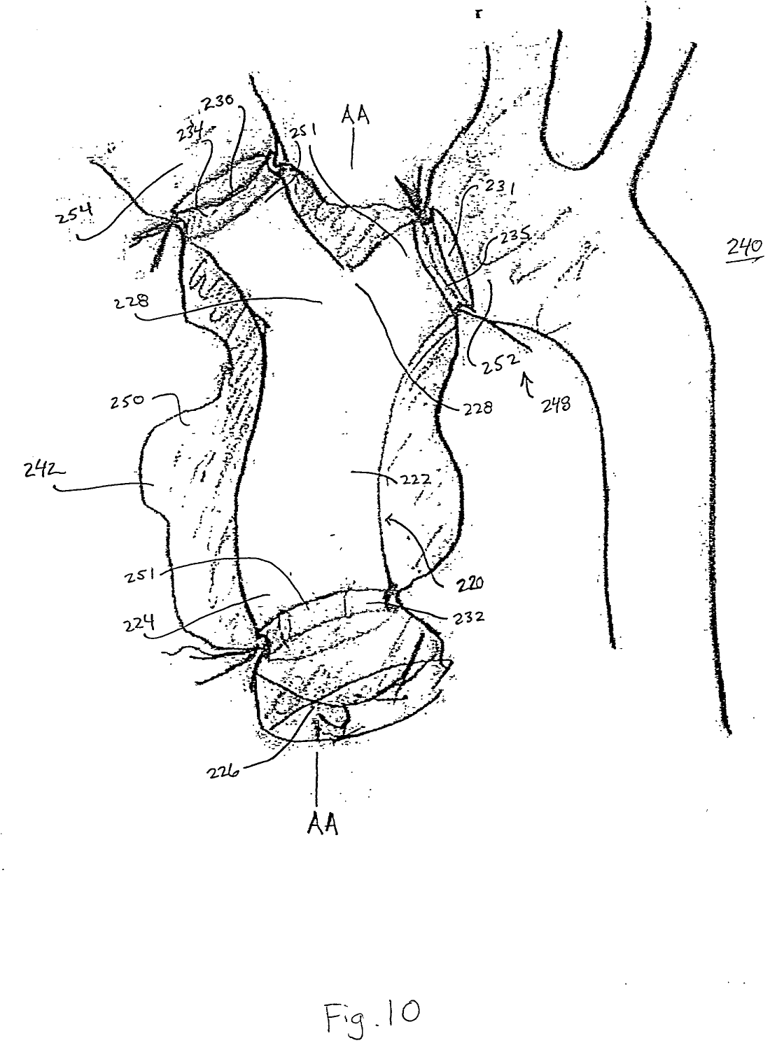

Referring to FIG. 10, an alternate embodiment of the intravascular prosthetic graft and related method is shown, similar to that described above with regard to FIGS. 1-9. An intravascular prosthetic graft 220 is provided for treatment of an aneurysm which includes a flexible tubular body 222 that is elongated and defines a longitudinal axis AA.

Flexible tubular body 222 has a first end 224 defining an opening 226. A second end 228 includes a first opening 230 and a second opening 231 which are similarly configured to opening 226.

A first ring member 232 is connected to first end 224 adjacent to opening 226 of first end 224. A second ring member 234 is connected to second end 228 adjacent to first opening 230 of second end 228. A third ring member 235 is connected to second end 228 adjacent to second opening 231 of second end 228. First ring member 232, second ring member 234 and third ring member 235 are integrally assembled with flexible tubular body 222 and have a substantially cylindrical configuration for conforming to the inner surface of a vessel. First ring member 232, second ring member 234 and third ring member 235 are designed to have a non-expanded state and are expandable to an expanded state, similar to that discussed above with regard to FIGS. 1-9.

The operation of intravascular prosthetic graft 220 during an aneurysmal treatment procedure will now be described. The surgical procedure described contemplates repairing aortic aneurysms by a minimal risk technique including repair of the ascending aorta, hemi-arch, as well as a portion of the innominate artery.

An arterial system 240 having a diseased vessel portion, such as, for example, an aneurysm 242 is shown. The method of repairing arterial system 240, such as, for example, treating aortic aneurysm 42 to repair an ascending aorta 250, a hemi-arch 252 and a portion of an innominate artery 254 includes the steps of providing an intravascular prosthetic graft 220, similar to those described above; determining polarity sites 251 for intravascular prosthetic graft 220 at the site of aneurysm 242; introducing intravascular prosthetic graft 220 to arterial system 240; delivering intravascular prosthetic graft 220 to the site of aneurysm 242; positioning intravascular prosthetic graft 220 for repairing a diseased portion of an aorta 248 at polarity sites 251; expanding intravascular prosthetic graft 220 to substantially conform to an inner surface of aorta 248; and fixing intravascular prosthetic graft 220 at polarity sites 251.

Similar to that described with regard to FIGS. 4-9, polarity sites 251 are determined, intravascular prosthetic graft 220 is introduced to arterial system 240 and intravascular prosthetic graft 220 is delivered to the site of aneurysm 242.

Intravascular prosthetic graft 220 is expanded to the expanded state, as discussed above, so that ring members 232, 234 and 235 engage the inner surface of aorta 248 such that intravascular prosthetic graft 220 engages aorta 248. Ring member 232, 234 and 235 are fixed to ascending aorta 250, innominate artery 254 and hemi-arch 252, similar to that described with regard to FIGS. 8 and 9, for arterial repair.

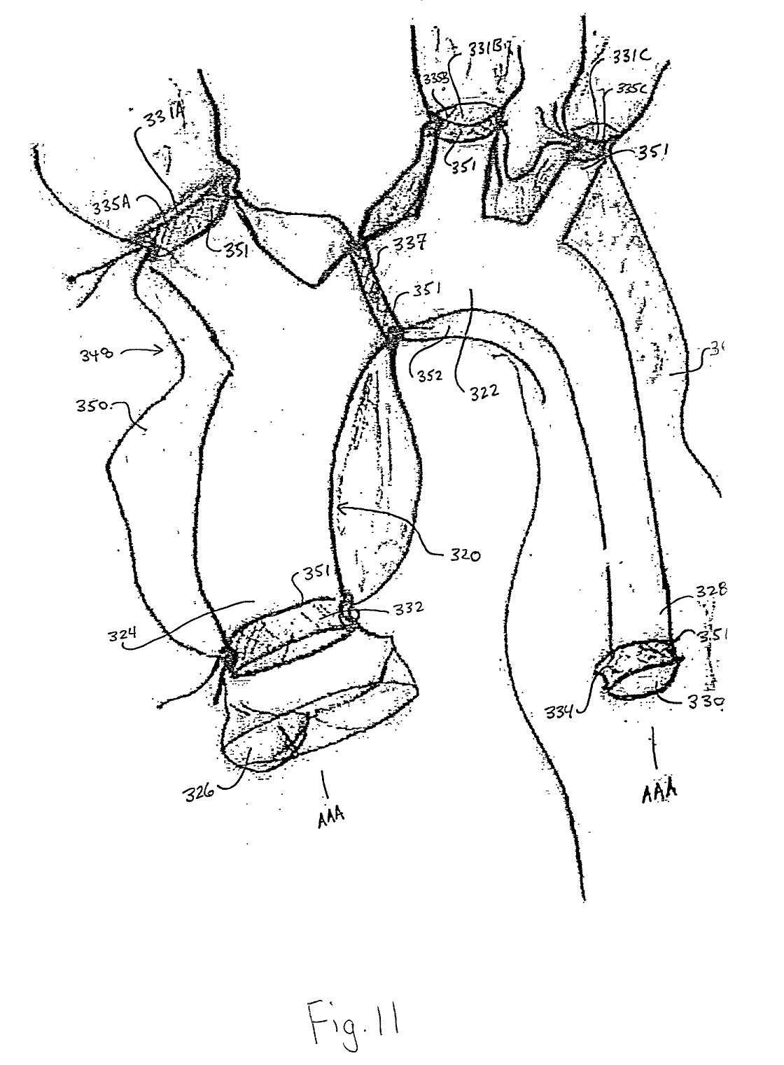

Referring to FIG. 11, another alternate embodiment of the intravascular prosthetic graft and related method is shown, similar to those described above. An intravascular prosthetic 320 is configured to repair an ascending aorta 350, an arch 352 of an aorta 348 and a portion of a descending aorta 362. Intravascular prosthetic graft 320 includes a flexible tubular body 322 that is elongated and defines a longitudinal axis AAA.

Flexible tubular body 322 has a first end 324 defining an opening 326. A second end 328 defines an opening 330 which is similarly configured. Flexible tubular body 322 has lateral openings 331A, 331B and 331C, which are similarly configured with openings 326 and 330.

A first ring member 332 is connected to first end 324 adjacent to opening 326 of first end 324. A second ring member 334 is connected to second end 328 adjacent to opening 330 of second end 328. A third ring member 335A, a fourth ring member 335B and a fifth ring member 335C are connected to intravascular prosthetic graft 320 adjacent openings 331A, 331B and 331C, respectively. A sixth ring member 337 is connected to intravascular prosthetic graft 320 between first end 324 and second end 328. Sixth ring member 337 may be variously spaced between first end 324 and second end 328.

First ring member 332, second ring member 334, third ring member 335A, fourth ring member 335B, fifth ring member 335C and sixth ring member 337 are integrally assembled with flexible tubular body 322 and have a substantially cylindrical configuration for conforming to the inner surface of a vessel. First ring member 332, second ring member 334, third ring member 335A, fourth ring member 335B, fifth ring member 335C and sixth ring member 337 are designed to have a non-expanded state and are expandable to an expanded state, similar to that discussed above.

The operation of intravascular prosthetic graft 320 during an aneurysmal treatment procedure will now be described. The surgical procedure described contemplates repairing aortic aneurysms by a minimal risk technique including repair of ascending aorta 350, arch 352 and a portion of descending aorta 362.

An arterial system 340 having a diseased vessel portion, such as, for example, an aneurysm 342 is shown. The method of repairing arterial system 340, such as, for example, treating aortic aneurysm 342 to repair ascending aorta 350, arch 352 and a portion of descending aorta 362 includes the steps of providing an intravascular prosthetic graft 320, similar to those described above; determining polarity sites 351 for intravascular prosthetic graft 320 at the site of aneurysm 342; introducing intravascular prosthetic graft 320 to arterial system 340; delivering intravascular prosthetic graft 320 to the site of aneurysm 342; positioning intravascular prosthetic graft 320 for repairing a diseased portion of an aorta 348 at polarity sites 351; expanding intravascular prosthetic graft 320 to substantially conform to an inner surface of aorta 348; and fixing intravascular prosthetic graft 320 at polarity sites 351.

Similar to that described above, polarity sites 351 are determined, intravascular prosthetic graft 320 is introduced to arterial system 340 and intravascular prosthetic graft 320 is delivered to the site of aneurysm 342.

Intravascular prosthetic graft 320 is expanded to the expanded state, as discussed above, such that ring members 332, 334, 335A, 335B, 335C and 337 engage the inner surface of aorta 348 such that intravascular prosthetic graft 320 engages aorta 348. Ring members 332, 334, 335A, 335B, 335C and 337 are fixed to ascending aorta 350, arch 352 and descending aorta 362, similar to that described above, for arterial repair.

It will be understood that various modifications may be made to the embodiments disclosed herein. Therefore, the above descriptions should not be construed as limiting, but merely as exemplification of the various embodiments. Those skilled in the art will envision other modifications within the scope and spirit of the present disclosure.

What is claimed is:

1. An intravascular prosthetic graft comprising:
a tubular body having a first end defining at least one opening and a second end defining at least one opening; and
at least one ring member being connected to the tubular body, the at least one ring member defining a surface about at least a portion thereof that is configured for receipt of ligature and a portion of a vessel therebetween, wherein the at least one ring member is adaptable from a first configuration to a second configuration.

2. An intravascular prosthetic graft as recited in claim 1, wherein the surface is configured as a groove.

3. An intravascular prosthetic graft as recited in claim 2, wherein the groove is substantially convex and formed in an outer surface of the at least one ring member about its circumference between ends thereof.

4. An intravascular prosthetic graft as recited in claim 1, whereby in the second configuration the at least one ring member is configured to engage an inner surface of a vessel such that the intravascular prosthetic graft forms a substantial seal with the inner surface of the vessel.

5. An intravascular prosthetic graft comprising:
a flexible elongated tubular body defining a longitudinal axis, the tubular body having a first end defining at least one opening and a second end defining at least one opening;
a first ring member being connected to the first end adjacent to the at least one opening of the first end; and
a second ring member being connected to the second end adjacent to the at least one opening of the second end, wherein the first ring member and the second ring member are adaptable from a first configuration to a second configuration.

6. An intravascular prosthetic graft as recited in claim 5, wherein the second ring member is oriented corresponding to the orientation of the at least one opening of the second end.

7. An intravascular prosthetic graft as recited in claim 5, wherein the at least one opening of the second end lies in a plane transverse to the longitudinal axis of the tubular body, the plane being angularly oriented relative to the longitudinal axis.

8. An intravascular prosthetic graft as recited in claim 5, wherein the first and second ring members have a substantially cylindrical configuration for conforming to the inner surface of a vessel.

9. An intravascular prosthetic graft as recited in claim 5, wherein the first configuration includes a non-expanded state such that the intravascular prosthetic graft is configured for introduction into a vessel.

10. An intravascular prosthetic graft as recited in claim 5, wherein the second configuration includes an expanded state such that the first and second ring members substantially conform to inner surfaces of a vessel.

11. An intravascular prosthetic graft as recited in claim 5, whereby in the second configuration the first and second ring members are configured to engage an inner surface of a vessel such that the intravascular prosthetic graft forms a substantial seal with the inner surface of the vessel.

12. An intravascular prosthetic graft comprising:
a flexible elongated tubular body defining a longitudinal axis, the tubular body having a first end defining an opening and a second end defining a first opening and a second opening;
a first ring member being connected to the first end adjacent to the opening of the first end;
a second ring member being connected to the second end adjacent to the first opening of the second end; and
a third ring member being connected to the second end adjacent to the second opening of the second end, wherein the first, second and third ring members are adaptable from a first configuration to a second configuration.

13. An intravascular prosthetic graft as recited in claim 12, whereby in the second configuration the first, second and third ring members are configured to engage an inner surface of a vessel such that the intravascular prosthetic graft forms a substantial seal with the inner surface of the vessel.

14. An intravascular prosthetic graft comprising:
a flexible elongated tubular body defining a longitudinal axis, the tubular body having a first end defining an opening, a second end defining an opening and first, second and third lateral openings being disposed on a lateral portion thereof;
a first ring member being connected to the first end adjacent to the opening of the first end;
a second ring member being connected to the second end adjacent to the opening of the second end;
a third ring member being connected to the first lateral opening;
a fourth ring member being connected to the second lateral opening;
a fifth ring member being connected to the third lateral opening; and
a sixth ring member being connected to the tubular body between the first end and the second end thereof, wherein the first, second, third, fourth, fifth and sixth ring members are adaptable from a first configuration to a second configuration.

15. An intravascular prosthetic graft as recited in claim 14, whereby in the second configuration the first, second, third, fourth, fifth and sixth ring members are configured to engage an inner surface of a vessel such that the intravascular prosthetic graft forms a substantial seal with the inner surface of the vessel.

16. A method of repairing at least a portion of an arterial system, the method comprising:
providing an intravascular prosthetic graft, the intravascular prosthetic graft including:
a tubular body having a first end defining at least one opening and a second end defining at least one opening, and
at least one ring member being connected to the tubular body, wherein the first ring member is adaptable from a first configuration to a second configuration;
determining a placement position for the intravascular prosthetic graft at the portion of an arterial system to be repaired;
introducing the intravascular prosthetic graft to the arterial system;
delivering the intravascular prosthetic graft to the placement position; and
adapting the at least one ring member from the first configuration to the second configuration to form a substantial seal with an inner surface of the arterial system.

17. A method of repairing at least a portion of an arterial system as recited in claim 16, further comprising the step of fixing the intravascular prosthetic graft to the inner surface of the arterial system at the placement position to form a substantial seal with the inner surface of the arterial system.
